(19) United States
(12) Patent Application Publication
(10) Pub. No.: US 2017/0183648 A1
(43) Pub. Date: Jun. 29, 2017

(54) METHOD, KIT AND ARRAY FOR FUNCTIONAL VALIDATION OF RESULTS DERIVED FROM HIGH THROUGHPUT STUDIES

(71) Applicant: Quick Biology, Inc., Arcadia, CA (US)

(72) Inventors: Wei LIAO, Northridge, CA (US); Yunguang TONG, Los Angeles, CA (US); Chen QING, Kunming (CN); Jian DING, Shanghai (CN)

(21) Appl. No.: 14/824,397

(22) Filed: Aug. 12, 2015

(51) Int. Cl. C12N 15/10; G06F 19/18

(52) U.S. Cl. CPC C12N 15/1086; C12N 15/1089; G06F 19/18

(57) ABSTRACT

A method of preparing a biomarker functional validation array includes selecting one or more lists of candidate agents, predicting functions of the one or more lists of candidate agents and combining the predicted functions to yield a final list of functions to be validated, and generating a biomarker functional validation array including agents targeting the final list of functions.

### BACKGROUND OF THE INVENTION

Field of the Invention

The present invention provides approaches that quickly and systematically evaluate biological functions of specific biological agents, such as biomarkers. This allows for quickly prioritizing and interpreting the results generated by high-throughput studies (such as studies of Next Generation Sequencing and microarray).

Discussion of the Related Art

Novel personalized medicine concepts (such as Precision Medicine) have been well accepted. Personalized medicine requires the use of biomarkers to determine the best treatment for a specific patient. To identify potential biomarkers, scientists use high throughput approaches.

The development of Next Generation Sequencing (NGS) and other high-throughput detection technology generated an unprecedented large amount of raw data. Analyses of these raw data resulted in massive amount of results, which create huge challenges to quickly determine the biological meaning (functions) of these results. Traditional concepts for biomarker investigation have reached a bottleneck.

Substantial work is wasted in biomarker research due to inefficient exploration of data from high throughput studies. The challenge is therefore not high throughput technologies, but the ways of selecting the promising candidates from the massive amount of results and determining the functions of selected biomarkers, so further investigation on biomarker development is justified.

Previous researches focus on validating the high throughput technique itself, rather than the function of the biomarkers. For example, WO2013/138727 A1 (method, kit and array for biomarker validation and clinical use) developed methods to validating microarray results using qPCR arrays. However, validating the technique of the high throughput studies is not sufficient in biomarker development. The most critical step is to understand the functions of the biomarker and related mechanisms. It therefore requires functionally validating the biomarker in a biological system.

There is therefore a need for a systematic solution for a quick functional validation of biomarkers, especially for the rapid increasing of newly identified genetic variants, such as mutations.

Platforms such as NGS, qPCR, ELISA have been used in biomarker investigation. Those platforms do not provide a systematic method for functional prediction and functional validation. Recently, functional genomics screening used siRNA or plasmid libraries to determine the contribution of a gene to certain phenotypes. These functional genomic screenings are not based on functional prediction and are therefore time-consuming, costly and generate unneeded data. Moreover, the majority of those using such platforms do not use knowledge databases to predict and select the functions, thus limiting their potential to select the best biomarker for further development.

### SUMMARY OF THE INVENTION

In embodiments, methods of prediction, selection and functional validation of biological active agents are provided. Suitably, the methods include selecting one or more lists of candidate agents, predicting functions of the lists of agents, selecting the predicted functions, and generating functional validation arrays to validate the predicted functions.

Suitably, the one or more lists of candidate agents are derived by analyzing one or more high-throughput studies. The one or more high-throughput study data sets are selected based on one or more of clinical utility, research interest, drug response, species and quality.

In embodiments, the analyzing of high-throughput studies includes analysis with one or more mathematical models selected from ANOVA analysis, t-test, survival analysis. In further embodiments, the analyzing includes combining agents from one or more of the mathematical models based on a desired classification implied by the data sets.

Suitably, the functional prediction further includes literature mining to predict the functions of one or more candidate agents.

Suitably, the predicted functions are selected using an algorithm to select the top ranked functions.

Suitably, the functional validation arrays comprise two major types: functional perturbation arrays or functional detection arrays. Functional perturbation array uses one or more functional perturbation agents to perturb one or more functions to evaluate the impact of these functions on a predetermine function. The functional perturbation array is good for understanding functional interactions, such as drug*drug, gene*drug, gene*gene interactions, or interaction of any bioactive agents. Functional detection array uses functional detection agent to detected altered functional changes caused by one or more bioactive agents.

Also provided are perturbation arrays prepared by the methods described herein, suitably where each defined location in the array corresponding to a perturbing agent for a biological target, which represents a biological function. In embodiments, the perturbation arrays are for perturbing the function of targeted biological agents, such as suppress or enhance kinases or enzyme activity, gene expression levels. Suitably, a perturbing agent can be a chemical agent, siRNA, miRNA, plasmid, protein, any other bioactive agent or combinations thereof. The outcome of the perturbation can be any predefined parameter which is measurable.

Also provided are detection arrays prepared by the methods described herein, suitably where each defined location in the array corresponds to a detection agent for a biological target, which represents a biological function. The functional detection array comprises qPCR array, immuno-PCR array, ELISA. In embodiments, the qPCR array is for analysis of messenger RNA (mRNA), or the qPCR array is for analysis of micro RNA (miRNA), or the qPCR array is for analysis of long non-coding RNA (IncRNA).

In embodiments, the immuno-PCR array is for analysis of protein or any biological target which can be detected by antibodies or other affinity probes. The immuno-PCR array is for analysis of protein levels, post translational modifications of proteins or combinations thereof.

In suitable embodiments, the arrays comprise one or more controls to assist data analysis and result interpretation. The controls include but not limited to array quality controls and normalization controls.

Also provided is an online platform to allow users to upload one or more candidate lists, predict functions and combine the prediction results, and generate functional validation arrays. When needed, the online platform allows user to upload data from high throughput studies and perform analyses to obtain candidate lists.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 shows the overall method of preparing a functional validation array as described herein.

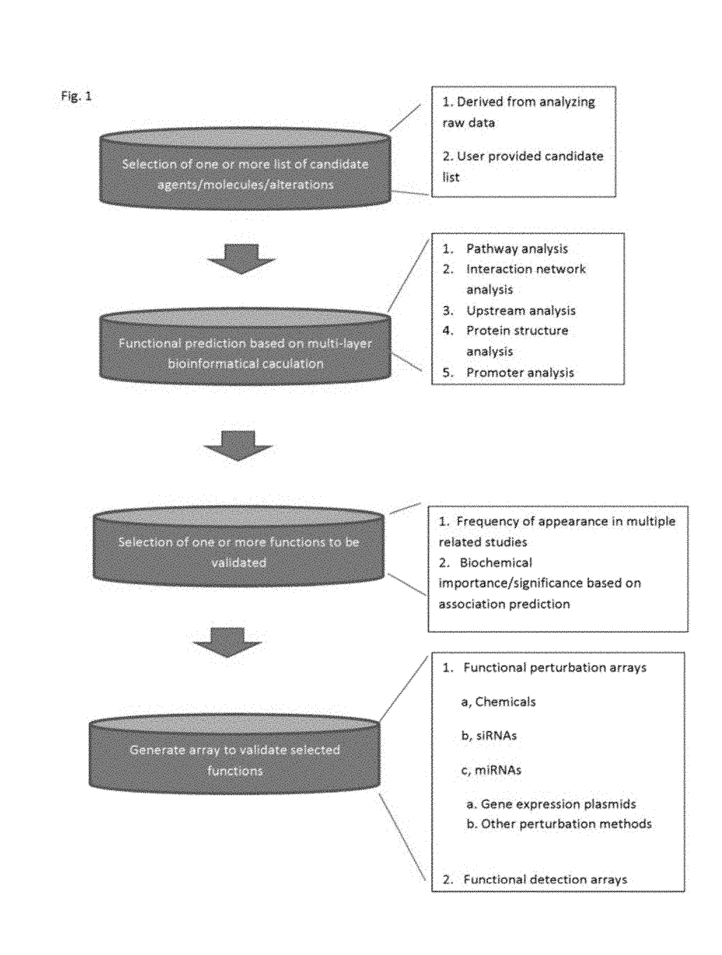

FIG. 2 shows an example of preparing and using a perturbation array as described herein.

FIG. 3 shows an example of preparing and using a detection array as described herein.

FIG. 4 shows an example of the online platform as described herein.

### DETAILED DESCRIPTION OF THE ILLUSTRATED EMBODIMENTS

Reference will now be made in detail to embodiments of the present invention, example of which is illustrated in the accompanying drawings.

The published patents, patent applications, websites, company names and scientific literature referred to herein are hereby incorporated by reference in their entireties to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter,

As used in this specification, the singular forms “a,” “an” and “the” specifically also encompass the plural forms of the terms to which they refer, unless the content clearly dictates otherwise. The term “about” is used herein to mean approximately, in the region of, roughly, or around, When the term “about” is used in conjunction with a numerical range, it modifies that range by extending the boundaries above and below the numerical values set forth.

In general, the term “about” is used herein to modify a numerical value above and below the stated value by a variance of 20%.

As used herein an “agent” refers to any materials that may have an effect on a biological system. Suitably, an agent refers to chemicals, genes, proteins, peptides, antibodies, cells, gene products, enzymes, hormones.

As used herein, a “biomarker” refers to a measurable characteristic that provides information of presence and/or severity of a disease or compromised state in a patient: the relationship to a biological pathway; a pharmacodynamic relationship or output; a companion diagnostic: a particular species; or a quality of a biological sample. Examples of biomarkers include genes, proteins, peptides, antibodies, cells, gene products, enzymes, hormones, etc,

Technical and scientific terms used herein have the meaning commonly understood by one of skill in the art to which the present application pertains, unless otherwise defined. Reference is made herein to various methodologies and materials known to those of ordinary skill in the art.

Since next generation sequencing (NGS), microarray technology and other molecular profiling became available for clinical specimens, thousands of disease-related biomarkers have been reported in the literature. However, the functions of these biomarkers are usually not well understood, which prohibit their further development.

Although bioinformatic analyses such as Gene Ontology and pathway analysis are able to predict functions of a list of biomarkers, these predictions are prone to be false positive. Neither academic researchers nor clinicians can make informed decision based on predicted function of a biomarker. Current validation of biomarker functions are low throughput and cannot meet the fast increasing results derived from NGS, microarray and other high throughput studies. Therefore it is an urgent need for a solution, which allows a quick validation of the predicted functions of biomarkers generated by the high throughput study.

A major proposal provided is that the validation of predicted functions is designed on an array in a format of microplates (for tissue culture or ELISA) and qPCR plates, which fits the normal workflow in most of biomedical labs. This approach will greatly facilitate ordinary labs to quickly validate functions of biomarkers.

Although the concept of using qPCR assay to validate NGS or microarray results has been demonstrated in the literature, massive validation the biomarker functions, especially with the guidance of functional prediction of bioinformatics, has not been demonstrated.

Also provided herein is a set of functional prediction algorithms that guide the qPCR array users to validate the function panel and eventually lead to a biomarker that fits the criteria to be further developed.

Ordinary functional genomic screening is costly and time consuming, requires expensive robots and professionals, which is suitable for conventional biomedical labs. By using functional prediction to select functions of interests, a scientist can reduce the functional validation experiments to a scale that can be handled by a conventional biomedical lab. This approach will eventually improve the overall efficiency of the research community.

Molecular detection methods such as ELISA and real time Polymerase Chain Reaction (PCR) are widely used in biomedical labs. Controls are critical to monitor the input difference between samples so that they can be compared equally. Controls also help to identify and minimize system variation. Choosing the wrong controls is one of the reasons for the failure to validate some of the “biomarkers” published in the literature. Critical controls to monitor assay quality itself are often neglected in the published literature, without which the systemic variation of the assay cannot be corrected for before the data are used for comparison. Provided herein is a description of how to choose the correct controls for various functional validation arrays.

The predicted functions of a biomarker are to be validated on a more practical assay platform, such as the ELISA, qPCR, immuno-PCR platform, in order to be accepted and justified for further development, or used in assisting clinical practice. Unfortunately, almost all “biomarkers” get stuck at the discovery phase and never have a chance to see their true practical use in the clinic, partially because the functions of the biomarkers are not well understood.

Provided herein is a systematic method to 1) select one or more candidate lists, which are derived from datasets of high throughput studies; 2) predict the functions of the selected one or more candidate lists; 3) select one or more predicted functions and generate functional validation array with optimized assay design and proper controls; and 4) provide a companion algorithm that will finalize the functional validation panels and generate a probability score for each function (ability to affect a pathway, phenotype or disease status) under study,

The kit components, functional validation array, described herein comprise two major types: 1) functional perturbation array and 2) functional detection array.

Functional perturbation array uses one or more functional perturbation agents to perturb one or more functions to evaluate the impact of these functions on a predetermine function. The functional perturbation array is good for understanding functional interactions, such as drug*drug, gene*drug, gene*gene interactions, or interaction of any bioactive agents.

Functional perturbation array is an array of pre-dispensed functional perturbation agents that are dried down on a plate. Each defined location within the array corresponds to a biological target (a kinase or any bioactive agents). The perturbation agents may be in a form of chemical, protein, siRNA, miRNA, plasmid and hormone. Detection can be via a report system, such as cell counting or an introduced reporter gene, depending on the function to be tested.

Functional detection array uses functional detection agent to detected altered functional changes caused by one or more bioactive agents.

Functional detection array is an array of pre-dispensed functional detection agents that are dried down on a plate. Each defined location within the array corresponds to a biological target. In the case of using qPCR to detection the functions, the target is a gene or any nucleic acid molecule. qPCR detection is via using an appropriate reaction mixture and biological and pathological samples (such as cDNA reverse-transcribed from total RNA).

Also provided herein is a system that also includes unique controls. A key issue in biological experiments is the control. The expression and function of any given gene can be affected by tissue type, disease status and sample collection and storage conditions. Even some common housekeeping genes can be altered by disease conditions. Using a panel of well-selected normalization controls, which better control the tissue sample amount used in each assay correctly, allows for an accurate comparison of the expression of certain genes is provided herein. The control panel also includes assay quality controls in order to help identify any condition that affects the evaluation of biomarker functions.

Functional perturbation agents can be chemical, siRNA, miRNA, plasmid or any other bioactive agents. These agents possess different property and require different delivery methods for optimized effects. Controls are required for delivery efficiency. Chemicals may have different solubility and therefore chemical with similar solubility will be dispensed to the same array and a vehicle control will be used as negative control. In the case of siRNA/miRNA, which requires transfection reagent like liposome to introduce the small molecules into cells, a control siRNA/miRNA labeled with fluorescence molecule will be used as a transfection control. Plasmid also required transfection reagent to enter into cells, a plasmid express a fluorescence protein will be used as a delivery control.

Functional detection agents can be PCR based nuclear acid detection or protein detection (Immuno-PCR), or antibody based protein detection (ELISA or immunobloting). In the functional detection arrays, negative controls are wells without detection agent. Detection agents targeting house-keeping genes, such as beta-actin, will be used as positive controls as well as normalization control.

If 96/384 plates are used to manufacture the functional validation arrays (either functional perturbation array or functional detection array), it is preferable that two arrays to be designed in the same 96/384 plate to minimize plate-to-plate variation.

Also provided is an online system that also includes a biomarker functional validation solution to allow for a customer to analyze their data of high throughput studies, predict functions, generate their functional validation array and analyze the validation results to select the best focus for further study.

It also provides a ranking system that can rank the predicted functions based on their importance when using them as biomarkers (for example the importance on a specific phenotype or a disease).

The methods provided focus on a quantitative molecular assay tool that systematically validates functions of biomarkers, with proper controls.

Also provided are methods to select predicted functions based on bioinformatics tools, including literature mining.

Public high-throughput analysis data sets are analyzed biologically, clinically and statistically for study topic and research subject, as well as data quality and sample grouping,

High-throughput analysis data set(s) with defined research topic(s) and good quality are processed to a standard that can be combined/compared and input into a bioinformatics model system(s).

Processed high-throughput analysis data are analyzed and ranked with well-established statistical model systems, such as t-test, ANOVA, survival test, association test, and regression modeling.

Research topics include disease classification, treatment response prediction, or pathway activation/inhibition. The research topics are used to mine the literature through publication databases in order to select the most important targets that studies have suggested play an important role in the defined topics as a marker. All the targets of interest are ranked based on their biomarker related importance.

Selected functions are combined by putting separate lists together or by re-ranking with the combination of all the different rankings, A final list (for example a 96-well or 384-well, depending on format) is generated by putting all of the most important predicted functions.

Provided herein is a system which includes afunctional validation array, which comprise functional perturbation array and functional detection array.

In the case of functional perturbation array, pre-dispensed and dried functional perturbation agents, each at a defined location within the array that focuses on well analyzed and selected biological targets (a gene or any molecule). Different treated samples are dispensed into each location to incubate with functional perturbation agents. Detection is via a predefined readout. In the case of using cell viability as readout, WST-1 reagent is added to each location to measure viable cells. In the case of luciferase reporter gene, agent to detection luciferase activity will be used.

In the case of qPCR-based functional detection array, pre-dispensed and dried PCR primers, each at a defined location within the array that focuses on well analyzed and selected biological targets (a gene or any nucleic acid molecule). Detection can be via qPCR using an appropriate reaction mixture and biological and pathological samples (such as cDNA reverse-transcribed from total RNA).

The assay for selected targets is designed and tested for its sensitivity, specificity and efficiency with an industry standard, the detection assay is specific, correlates well with input change and is sensitive enough for low signal detection.

Also provided is a system which also includes a functional prediction platform to allow for customer to predict the function of their candidate list online and select the functions to be validated. The platform allows customers to place an order to generating functional validation arrays to validate the predicted functions. The systems disclosed herein provide QC analysis to help customers evaluate functional validation assay quality, the sample quality and potential outliers.

The systems disclosed herein provide a ranking system that can rank the functions based on their importance when selecting them as functional related target (for example the frequency of appearance in multiple predictions).

In embodiments, high-throughput gene expression data sets are selected based on research interest, study objective, species and quality.

Selected data sets are normalized and then analyzed by t-test, ANOVA, association analysis and to generate candidate lists. Top-ranked targets from functional analyses are combined to produce a list of predicted functions. Functional detection assay for all candidate targets are designed and tested for technical sensitivity, specificity, and dynamic range.

Appropriate normalization control assays and performance controls are added to complete the final functional validation assay.

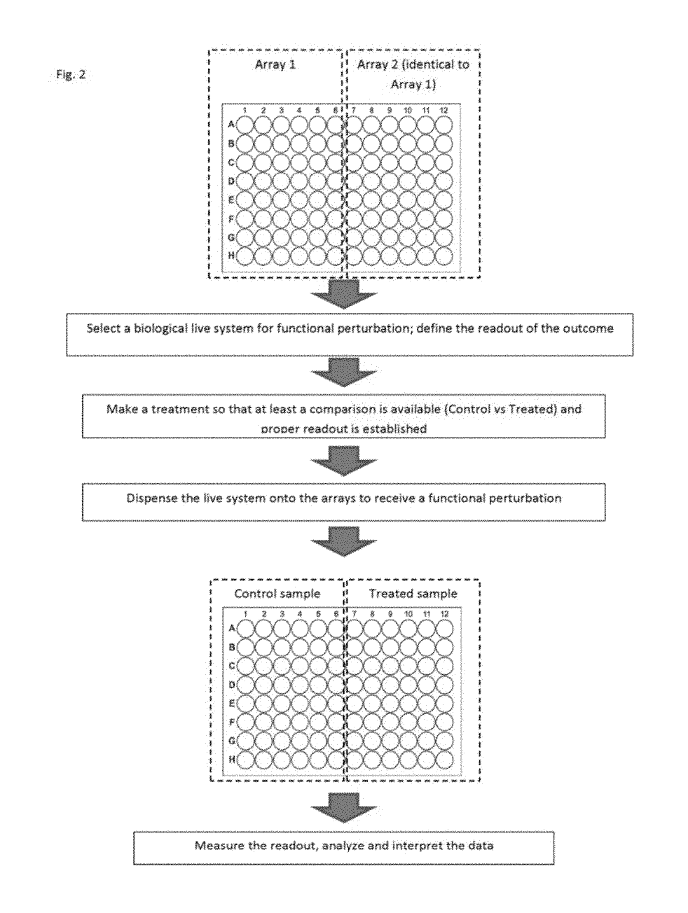

FIG. 2 shows an example of preparing and using functional perturbation array. In this case, 96 plates are used and the arrays are on the same plate to minimize plate to plate variation. Researcher's efforts: 1) build an experimental system, which allows further functional perturbation; define readout of outcome; collect samples and dispense to functional perturbation arrays, then 2) incubation and measure the designated signals, 3) Shows Data analysis portal:

Normalization of detected signal, with final normalization control selected based on researcher's samples,

A. Normalization of detected signal, with final normalization control selected based on researcher's samples,

B. Ranking of target functions for their difference between the two or multiple samples.

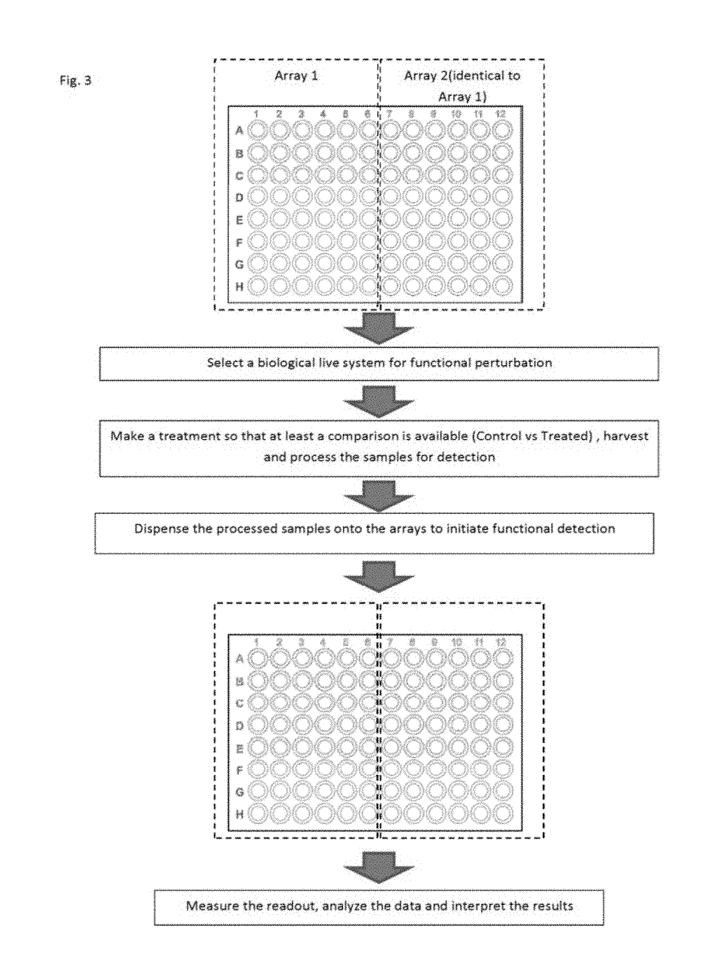

FIG. 3 shows an example of preparing and using functional detection array. In this case, 96 plates are used and the arrays are on the same plate to minimize plate to plate variation. Researcher's efforts: 1) build an experimental system, collect and process samples to make it suitable for detection and dispense to functional detection arrays, then 2) incubation and measure the designated signals, 3) Shows Data analysis portal:

A. Normalization of detected signal, with final normalization control selected based on researcher's samples,

B. Ranking of target functions for their difference between the two or multiple samples.

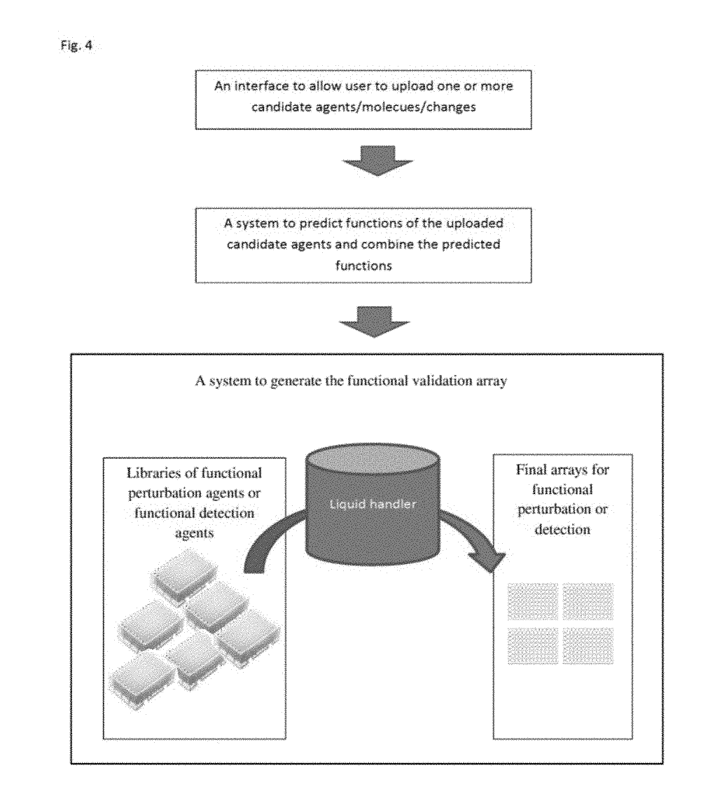

FIG. 4 shows an example of online platform preparing functional validation arrays. In this case, the system provide an interface to allow researcher upload one or more list of candidate agents directly, or allow research to upload data of high throughput studies to obtain one or more list of candidate agents through analyses. The online system allows researchers to predict the functions of the candidate agents and select functional to be validated. The online system will control a liquid handler to take functional perturbation agents or functional detection agents from libraries of functional validation agents and dispense it into arrays, suitably microplates.

Development of Biomarker Functional Validation Array

In embodiments, methods of preparing a functional validation array are provided. Suitably, such methods comprise selecting one or more candidate agent lists derived from high-throughput study data sets, predicting the functions of the one or more candidate agent lists by one or more mathematical models to yield predicted functions, selecting functions to be validated, generating the functional validation array, either through perturb or detect the selected functions, name as functional perturbation array or functional detection array, respectively.

In embodiments, the one or more high-throughput data sets (including microarray data sets and next generation sequencing) are selected based on one or more of clinical utility (e.g. disease specific biomarkers), research interest (e.g., biological pathway-specific biomarkers), drug response (e.g., pharmacodynamic biomarkers or companion diagnostic biomarkers), species and quality.

In embodiments, the analyzing comprises analysis of the data sets with one or more mathematical models including but not limited to t-test, ANOVA, association analysis.

In embodiments, the functional analysis comprises use of two, or more suitably, on the data to generate the predicted functions and the function validation array. Suitably, the functional analysis comprise the pathway analysis to predict the effect of a biomarker or drug on a pathway, interaction network analysis to predict the effect of protein-protein interaction, promoter analyses to predict the effects of transcription factors on a gene promoter or the effect of a transcription factor on downstream genes, 5′ UTR (untranslated region) analysis to predict the effects of microRNAs on translation or mRNA stability of a gene, the effect of a miRNA on potential targets, or upstream analysis to predict the potential upstream regulators of a given list of genes.

As described herein, the analysis can further comprise literature mining to yield the predicted functions. This allows for the addition of further information to clarify and define the desired function predictions.

Suitably, the methods further comprise selecting one or more controls for inclusion of control functions in the functional validation array. As described herein, it is the selection of these control agents (i.e., agents that do not demonstrate a change in a functional validation array or agents always demonstrate a change in the functional validation array) that provides one of the unique agents of the methods and arrays provided herein, so as to produce the most useful array information.

Also provided are functional validation arrays prepared by the methods described herein. The functional validation array comprises two major types: functional perturbation array and functional detection array.

For functional perturbation array, each defined location in an array is used to perturb a biological function.

In embodiments, the functional perturbation arrays are designed for perturbed various functions to see the effects of these perturbed functions on the outcome. The perturbation targets including various pathways, enzymes, transcription factors, cell status.

The perturbation agents can be any bioactive agent, which include, but not limited to chemicals, siRNA, miRNA, proteins, peptide and hormones. Correspondingly, the perturbation array may be an array of any bioactive agents.

The designed functional perturbation assay is evaluated with control samples for its performance (including efficiency of delivery of perturbation agents, etc.).

In the case of chemical-based functional perturbation, assay performance controls include negative controls, which are vehicle solutions; and positive controls, which can be fluorescence chemicals to allow monitoring under a fluorescence microscope or microplate reader.

In the case of siRNA/miRNA/plasmid-based functional perturbation, assay performance controls include negative controls, which are transfection reagents only; and transfection controls, which fluorescence labeled siRNA/miRNA or plasmid expressing GFP.

For functional detection array, each defined location in an array is used to detect a biological function.

In embodiments, the functional detection arrays are designed for detection of various functions, including the effects on various pathways, phenotypes, for example, for analysis of cell cycle control, for analysis of EGF pathway, for analysis of cell death, etc. as well as combinations thereof.

The methods then further comprise assigning a single probability score to the function detection. That is, a single value is assigned to the detection that can be utilized to determine whether or not the level of detection is indicative of the measured/desired outcome. The “cut-off” value for a detected function the probability score below or above which the presence of a detected function is determinative—is suitably scalable, i.e., up or down as desired,

As described herein, the focus of functions to be validated can be selected based on market needs, customer request, collaboration, etc.

High-throughput study is selected based on the topic (from public database or from collaboration or a customer's own data). Data is normalized and suitably annotation file is downloaded. The normalized data is used for analysis. T-test, anova, association analysis are used to identify related genes and generate an independent list. All the lists are combined based on each gene's ranking in each list,

Literature mining is used to find well-accepted, publically recognized biomarker functions of similar kind and added to the functions list.

In the case of qPCR based detection, gene target sequences are put into a primer design tool for assay design. Probe(s) are designed, and a qPCR primer pair is designed around each probe design. Suitably, an assay design set including a pair of primer and a probe, are used.

The designed functional detection assay is evaluated with control samples for its performance (including sensitivity, specificity, efficiency, etc.).

In the case of functional detection at mRNA levels, such as RT-qPCR, assay performance controls include genomic DNA contamination controls, reverse transcription efficiency controls and qPCR performance controls to aid in identification of any low quality data.

In the case of functional detection at protein levels, such as immunoPCR, ELISA, immunoblot, array control include internal control (such as house-keeping genes like beta-actin) for normalization and background control (a location dispensed with vehicle control solution without any functional detection agents).

Use of Biomarker Functional Validation Array

The functional perturbation array is useful to detection the effects of multiple perturbation agents on a specific function. For functional perturbation array, a live model system such as cell will be dispensed onto array to allow further functional perturbation by the dispensed agent on the array. In the case of using cells, two cell types will be generated from the same mother cells, one is control and the other is introduced with a biomarker or a function reporter. The control and treated cells are dispensed onto two identical arrays for incubation with perturbation agents. Suitably the two identical arrays are in the same 96/384 plates. After perturbation, the affected functions will be detected with a predefined reporter.

For functional detection array, detections of multiple functions will be performed. A live model system will be used. In the case of using cells, two cell types will be generated from the same mother cell, one is control and the other one is introduced with a biomarker. The cell lysate of the cells will be obtained and incubated with detection agents on the array. Detection will be performed by a uniform reaction, such as PCR, Horseradish peroxidase (HRP) colorimetric reaction.

It will be readily apparent to one of ordinary skill in the relevant arts that other suitable modifications and adaptations to the methods and applications described herein can be made without departing from the scope of any of the embodiments. It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

What is claimed is:

1. A method of preparing a biomarker functional validation array, comprising:
a. selecting one or more lists of candidate agents;
b. predicting functions of the one or more lists of candidate agents and combining the predicted functions to yield a final list of functions to be validated; and
c. generating the biomarker functional validation array including agents targeting the final list of functions.

2. The method of claim 1, wherein the one or more lists of candidate agents are derived from analyzing one or more high throughput studies.

3. The method of claim 1, wherein the one or more lists of candidate agents are selected based on one or more selected from the group consisting of research interest, clinical utility, drug response, species and quality.

4. The method of claim 1, wherein the predicting functions comprises an analysis with one or more methods selecting from the group consisting of promoter analysis, 5′ UTR analysis, pathway analysis, interaction network analysis, upstream analysis and literature mining.

5. The method of claim 1, wherein the biomarker functional validation array comprises a functional perturbation array, a functional detection array, and a protocol to use the functional validation array.

6. A functional perturbation array prepared by the method of claim 5.

7. The perturbation array of claim 6, where each defined location in the perturbation array corresponds to an agent perturbing a biological function.

8. The perturbation array of claim 6, wherein the perturbation array is adapt for increasing or decreasing of messenger RNA (mRNA), micro RNA (miRNA), long non-coding RNA (IneRNA), protein, modifications of protein, metabolites, or combinations thereof.

9. The perturbation array of claim 6, wherein the perturbation array comprises perturbation agents including a chemical agent, siRNA, miRNA, plasmid, protein, metabolite or combinations thereof.

10. A functional detection array prepared by the method of claim 5.

11. The detection array of claim 10, wherein each defined location in the detection array corresponds to an agent detecting activation level of a biological function.

12. The detection array of claim 10, wherein the detection array is for analysis of messenger RNA (mRA), micro RNA (miRNA), long non-coding RNA (IneRNA), protein, modifications of protein, metabolites or combinations thereof.

13. The detection array of claim 10, wherein the detecting includes qPCR or immuno-PCR based detection.

14. An online platform to implement the method of claim 1, comprising:
a. an interface to allow user to upload one or more lists of candidate agents;
b. a system to predict functions of the uploaded candidate agents and combine the predicted functions to yield a final list of functions to be validated; and
c. a system to generate the biomarker functional validation array comprising validation agents targeting the final list of functions.

15. The online platform of claim 14, wherein the online interface further comprises an online data analysis system to allow user to obtain one or more lists of candidate agents through uploading and analyzing data of one or more high throughput studies.

16. The online data analysis system of claim 15, further comprising data analysis pipelines to perform analyses of one or more selected from the group consisting of microarray data, RNA sequencing data, Exomic sequencing data, Whole genome sequencing data, proteomics data, and metabonomics data.

17. The online platform of claim 14, wherein the system to predict functions comprises function prediction algorithms including sequence and structure analysis for DNA, RNA and protein, pathway analysis, interaction network analysis, and upstream regulator analysis.

18. The online platform of claim 14, wherein the system to generate the biomarker functional validation array further comprises at least a library of functional validation agents and a liquid handling instrument to dispense the selected functional validation agents into arrays.
